(19) United States
(12) Patent Application Publication
(10) Pub. No.: US 2026/0335607 A1
(43) Pub. Date: Oct. 29, 2026

(54) Braided Reinforced Tearaway Sheath Structure and Manufacturing Method Thereof

(71) Applicant: Ningbo Linstant Polymer Materials Co., Ltd., Ningbo City (CN)

(72) Inventors: Xiaobo SONG, Ningbo City (CN); Zhiyuan QIAO, Ningbo City (CN); Xiaopeng LI, Ningbo City (CN); Rui ZHANG, Ningbo City (CN); Pengjie ZHANG, Ningbo City (CN)

(73) Assignee: Ningbo Linstant Polymer Materials Co., Ltd., Ningbo City (CN)

(21) Appl. No.: 19/399,619

(22) Filed: Nov. 24, 2025

(30) Foreign Application Priority Data
Mar. 20, 2025 (CN) 202510329087.5

(51) Int. Cl. A61M 25/06; A61M 25/00

(52) U.S. Cl. CPC A61M 25/0668; A61M 25/0012; A61M 25/0023; A61M 25/0045; A61M 25/005; A61M 2025/0047; A61M 2205/0222; A61M 2207/00

(57) ABSTRACT

The present invention discloses a braided reinforced tearaway sheath structure and a manufacturing method thereof, comprising a sheath body. The sheath body includes a sleeve portion and a guiding insertion portion provided at the head end of the sleeve portion. A connector is provided at the tail end of the sleeve portion. The sleeve portion includes an inner layer tube, a braided tube, and an outer layer tube sequentially arranged from the inside to the outside. The sheath body is provided with a plurality of tear grooves along the length direction, and both ends of each tear groove extend to both ends of the sheath body, and the sheath body is further sealed and wrapped with at least one layer of tearable film. It has the effect of improving the strength of the tearaway sheath structure and making the tearing process of the tearaway sheath safer.

### TECHNICAL FIELD

The present invention relates to the technical field of medical sheaths, and more particularly to a braided reinforced tearaway sheath structure and a manufacturing method thereof.

### BACKGROUND

A tearaway sheath (also known as a tear sheath or a cutting sheath) plays an important role in cardiovascular interventional procedures, especially in emergency situations where rapid entry and exit of blood vessels are required. The design of this type of sheath is intended to provide rapid and convenient operation, and to ensure protection of blood vessels during the procedure.

Existing tearaway sheaths are typically composed of multi-layer tube structure components, including an inner layer, an intermediate reinforcing layer, and an outer layer, each layer having a specific function, such as the inner layer providing lubricity, the intermediate layer providing support, and the outer layer providing protection. Tearaway sheaths on the market typically have a pre-embedded tear line on the tube wall of the sheath, and at the same time, the structural strength is reduced near the tear line. When it is necessary to urgently remove the interventional device in the sheath, the sheath can be torn open using the tear line, thereby achieving the purpose of quickly removing the interventional device. To this end, the inner layer, intermediate reinforcing layer, and outer layer of the existing tearaway sheaths are all made of tearable, low-strength polymer materials to meet the purpose of rapid tearing. This will inevitably reduce the overall structural strength of the sheath, leading to sheath fracture during interventional procedures. In addition, the tear line is prone to lacerating the skin during the process of pulling the tear line to tear the sheath, which is not safe and needs to be improved.

### SUMMARY

The purpose of the present invention is to provide a braided reinforced tearaway sheath structure and a manufacturing method thereof, which has the effects of improving the structural strength of the tearaway sheath and making the tearing process of the tearaway sheath safer.

The above technical purpose of the present invention is achieved through the following technical solutions: A braided reinforced tearaway sheath structure is provided, comprising a sheath body, wherein the sheath body comprises a sleeve portion, a guiding insertion portion provided at the head end of the sleeve portion, and a connector provided at the tail end of the sleeve portion.

The sleeve portion comprises an inner tube, a braided tube and an outer tube sequentially arranged from the inside to the outside, and the sheath body is provided with a plurality of tear grooves along the length direction. Each of the tear grooves extends to both ends of the sheath body, and the sheath body is further sealed and wrapped with at least one layer of tearable film.

In this embodiment, the sheath body can be smoothly inserted into a predetermined position through the guiding insertion portion at its head, and then an external medical device may be connected by using the connector, so that the interventional device can be inserted into the sheath body and accurately guided into a predetermined position. During the process of placing the tearaway sheath into the predetermined position, the tearaway sheath may be bent. At this time, the braided tube can improve the flexibility and anti-torsion performance of the tearaway sheath, thereby improving the structural strength of the tearaway sheath and preventing the tearaway sheath from breaking due to excessive bending. At the same time, during the placement of the tearaway sheath, the outermost film can prevent the surface of the sheath from scratching the skin; in addition, when the interventional device needs to be quickly removed from the tearaway sheath in an emergency, the operator can directly tear the sheath body, so that the film can be gradually torn from the tail end towards the head end of the tearaway sheath with the tear groove as a dividing line, thereby directly exposing the interventional device. In contrast to existing solutions where the sheath is easily torn and the skin is strained by tearing lines, the present invention eliminates the tearing line, and the user can directly tear the tearaway sheath through the tear groove. The tearing process will not cause skin strain due to the tearing line, and it is safer to use. The net effect is improvement of structural strength of the tearaway sheath while making the tearing process of the tearaway sheath safer.

In an embodiment, the inner side of the tear groove is in communication with the inner cavity of the sleeve portion, and the outer side of the groove extends through to the surface of the sheath body.

In this embodiment, the tear groove is configured such that the structural strength of the tearaway sheath near the tear groove is lower, and the operator can more easily tear the tearaway sheath along the tear groove.

In an embodiment, the tear groove is spirally arranged around the surface of the sheath body.

In this embodiment, the spirally arranged tear groove enables the sheath body to have better bending resistance performance, which can effectively prevent the tearaway sheath from breaking due to excessive bending.

In an embodiment, the tear groove is linearly arranged on the surface of the sheath body.

In this embodiment, the operator can tear the sheath body into several straight strips through the straight tear groove, which is convenient for taking the interventional device out of the tearaway sheath.

In an embodiment, the outer diameter of the guiding insertion portion gradually decreases from the end close to the sleeve portion to the end away from the sleeve portion.

In this embodiment, the end of the guiding insertion portion is conically arranged, which can reduce the resistance during the insertion of the tearaway sheath, thereby reducing the damage to the skin tissue.

In an embodiment, the connector is set as a locking cap for externally connecting a dilator.

In this embodiment, the design of the locking cap can improve the firmness and sealing degree of the connection between the sheath body and the dilator.

In an embodiment, the film comprises a tubular polymer heat-shrinkable film, and the material of the polymer heat-shrinkable film comprises any one of TPE, PO, PVC, PEBAX, PET or silicone rubber.

In an embodiment, the material of the inner tube comprises a self-lubricating polymer, such as any one of PTFE, PEEK, PE, POM or PI, the intermediate braided tube is three-dimensionally braided by a plurality of fiber filaments outside the inner tube, the material of the fiber filaments comprises any one of polymer wire or metal wire, and the outer tube comprises a thermoplastic polymer material.

Another technical purpose of the present invention is to provide a manufacturing method of a braided reinforced tearaway sheath structure, comprising the following steps:
S1: Inner tube preparation: alkali washing the surface of the inner tube to remove oil stains on the surface;
S2: Preparing the braided tube: three-dimensionally braiding a plurality of fiber filaments outside the inner tube through a three-dimensional braiding machine to form the braided tube;
S3: Preparing the outer tube: welding the outer tube outside the inner tube braided with the braided tube by hot melting or heat shrinking, and compositing to form the sheath body;
S4: Processing the tear groove: cutting the tear groove on the surface of the sheath body by high-precision mechanical cutting technology or laser cutting technology; S5: Film coating: tightly wrapping the film on the outside of the sheath body formed with the tear groove in S4, and forming the final product.

In this embodiment, a tear groove is opened on the surface of the composite assembled sheath body, and then the film is tightly wrapped outside the sheath body with the tear groove opened, and the sheath body is radially limited by using the film, so that the tearaway sheath can have sufficient bending support in the radial direction.

In an embodiment, in step S5, the film is fixedly connected to the surface of the sheath body by any one of bonding, dip coating or welding.

In summary, the present invention has the following beneficial effects:

By adopting the tear groove opened in the length direction of the sheath body, the sheath body comprises an inner tube, an intermediate braided tube and an outer tube, and the sheath body comprises a sleeve portion and a guiding insertion portion provided at the head end of the sleeve portion. During use, the sheath body can be smoothly inserted into a predetermined position through the guiding insertion portion at its head, and then an external medical device is connected by using the connector, so that the interventional device can be inserted into the sheath body and accurately guided into the predetermined position. During the process of placing the tearaway sheath into the predetermined position, the tearaway sheath may be bent. At this time, the braided tube can improve the flexibility and anti-torsion performance of the tearaway sheath, thereby improving the structural strength of the tearaway sheath and preventing the tearaway sheath from breaking due to excessive bending. At the same time, during the placement of the tearaway sheath, the outermost film can prevent the surface of the sheath from scratching the skin. Additionally, when the interventional device needs to be quickly removed from the tearaway sheath in an emergency, the operator can directly tear the sheath body, so that the film can be gradually torn from the tail end towards the head end of the tearaway sheath with the tear groove as a dividing line, thereby directly exposing the interventional device. In contrast to existing solutions where the sheath is easily torn and the skin is strained by tearing lines, the present invention eliminates the tearing line, and the user can directly tear the tearaway sheath through the tear groove. The tearing process will not cause skin strain due to the tearing line, and is safer to use, which has the effect of improving the structural strength of the tearaway sheath and making the tearing process of the tearaway sheath safer.

### BRIEF DESCRIPTION OF THE DRAWINGS

FIG. 1 is an overall view of the present invention.

FIG. 2 is an overall structural diagram of specific embodiment one of the present invention, wherein the covering film is not shown.

FIG. 3 is an overall structural diagram of specific embodiment two of the present invention, wherein the covering film is not shown.

FIG. 4 is a longitudinal sectional view of FIG. 3 of the present invention.

FIG. 5 is a partially enlarged view of area A in FIG. 4 of the present invention.

Reference numbers: 1, sheath body; 2, sleeve portion; 21, inner layer tube; 22, braided tube; 23, outer layer tube; 3, guiding insertion portion; 4, connector; 5, covering film; 6, tear groove.

### DETAILED DESCRIPTION OF EMBODIMENTS

The present invention will be further described below with reference to the accompanying drawings.

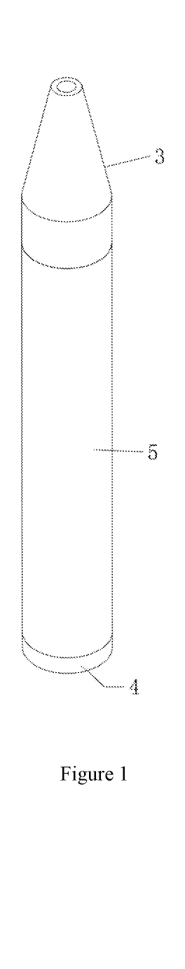

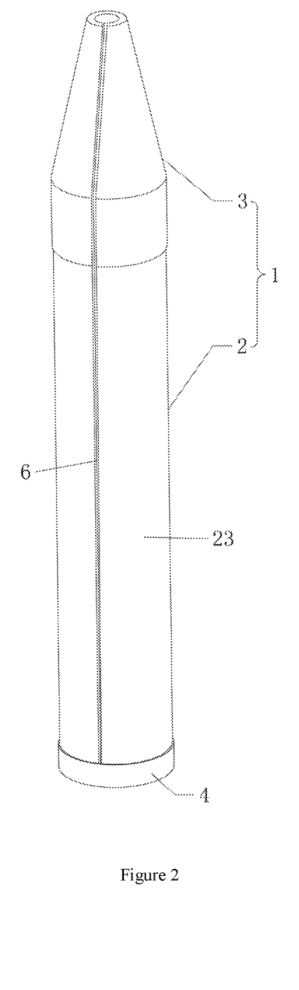

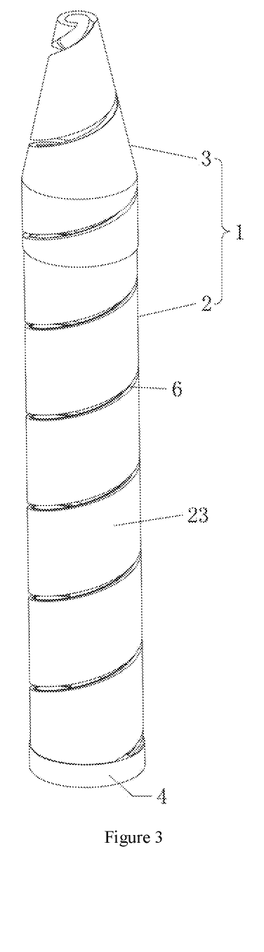

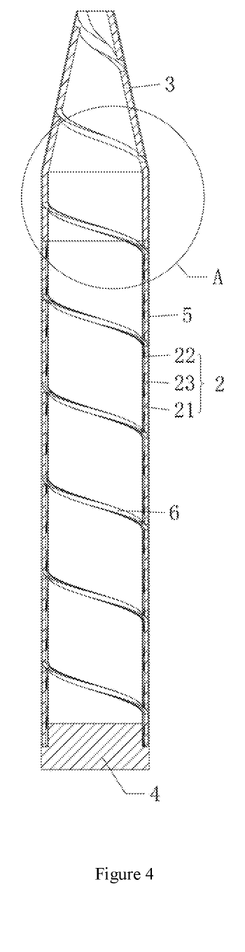

Embodiment 1: A braided reinforced tearable sheath structure, as shown in FIGS. 1-4, includes a sheath body 1. The sheath body 1 includes a sleeve portion 2 and a guiding insertion portion 3 disposed at the head end of the sleeve portion 2. The tail end of the sleeve portion 2 is provided with a connector 4. The sleeve portion 2 includes an inner layer tube 21, a braided tube 22, and an outer layer tube 23, which are sequentially arranged from the inside to the outside. The sheath body 1 is provided with a plurality of tear grooves 6 along the length direction. Both ends of each tear groove 6 extend to both ends of the sheath body 1, and the sheath body 1 is further sealed and wrapped with at least one layer of tearable film 5. In this embodiment, the thickness of the film 5 is controlled between 0.1-2 mm, and the tear strength of the film 5 is controlled between 0.5-10 N/mm. The inner side of the tear groove 6 is in communication with the inner cavity of the sleeve portion 2, and the outer side of the tear groove 6 penetrates to the surface of the sheath body 1. The penetrating tear groove 6 makes the structural strength of the tearable sheath lower near the tear groove 6, and it is easier for the operator to tear the tearable sheath along the tear groove 6. The tear grooves 6 are spirally arranged around the surface of the sheath body 1, and the spiral tear line is arranged as a conical spiral line on the guiding insertion portion 3. The spirally arranged tear grooves 6 enable the sheath body 1 to have better bending resistance, which can effectively prevent the tearable sheath from being broken due to excessive bending. The outer diameter of the guiding insertion portion 3 gradually decreases from the end close to the sleeve portion 2 toward the end away from the sleeve portion 2, so that the end of the guiding insertion portion 3 is conically arranged, which can reduce the resistance during the insertion of the tearable sheath, thereby reducing damage to skin tissue. The connector 4 is designed as a locking cap for externally connecting a dilator. The design of the locking cap can improve the firmness and sealing degree of the connection between the sheath body 1 and the dilator. The film 5 is made of a tubular polymer heat-shrinkable film, and the material of the polymer heat-shrinkable film is any one of TPE, PO, PVC, PEBAX, PET or silicone rubber. The material of the inner layer tube 21 is a self-lubricating polymer, such as any one of PTFE, PEEK, PE, POM or PI.

The intermediate braided tube 22 is three-dimensionally braided by a plurality of fiber filaments outside the inner layer tube 21, and the material of the fiber filaments is any one of polymer wire or metal wire. The outer layer tube 23 is made of a thermoplastic polymer material.

The basic working principle of the present invention is as follows: tear grooves 6 are opened in the length direction of the sheath body 1. The sheath body 1 includes an inner layer tube 21, an intermediate braided tube 22, and an outer layer tube 23, and the sheath body 1 includes a sleeve portion 2 and a guiding insertion portion 3 disposed at the head end of the sleeve portion 2. During use, the sheath body 1 can be smoothly inserted into a predetermined position through the guiding insertion portion 3 at its head, and then the external medical device is connected by the connector 4, so that the interventional device can be inserted into the sheath body 1 and accurately guided into the predetermined position. During the process of placing the tearable sheath into the predetermined position, the tearable sheath may be bent. At this time, the braided tube 22 can improve the flexibility and anti-torsion performance of the tearable sheath, thereby improving the structural strength of the tearable sheath and preventing the tearable sheath from being broken due to excessive bending. At the same time, during the placement of the tearable sheath, the outermost film 5 can prevent the sheath surface from scratching the skin. In addition, when the interventional device needs to be quickly removed from the tearable sheath in an emergency, the operator can directly tear the sheath body 1, so that the film 5 can be gradually torn from the tail of the tearable sheath toward the head along the tear groove 6 as a dividing line, thereby directly exposing the interventional device. Different from the existing situation that the sheath tube is easily torn and the skin is strained through the tear line, the present invention cancels the tear line, and the user can directly tear the tearable sheath through the tear groove 6. The tearing process will not cause the tear line to strain the skin, making it safer to use, and has the effect of improving the structural strength of the tearable sheath and making the tearing process of the tearable sheath safer.

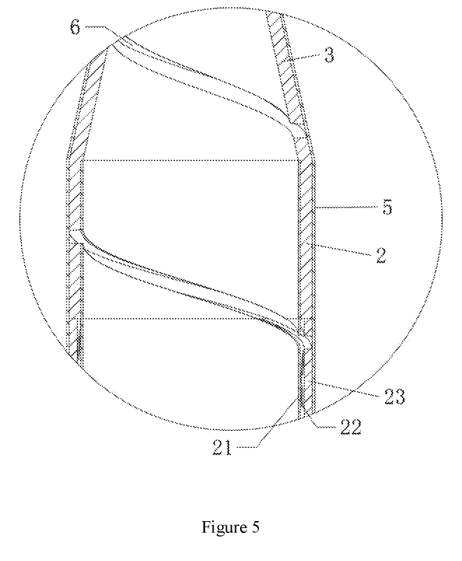

Embodiment 2: A braided reinforced tearable sheath structure, as shown in FIG. 5. The difference between this embodiment and the specific embodiment 1 is that the tear grooves 6 are linearly arranged on the surface of the sheath body 1. Different from the spiral tear grooves 6, the linear tear grooves 6 are arranged so that the operator can tear the sheath body 1 into several straight strips through the linear tear grooves 6, which is more convenient for the operator to take out the interventional device from the tearable sheath.

The other structures of this embodiment are the same as those of the specific embodiment 1, and will not be repeated here.

Another technical object of the present invention is to provide a method for manufacturing a braided reinforced tearable sheath structure, including the following steps:
S1: Preparation of the inner layer tube 21: alkali washing the surface of the inner layer tube 21 to remove oil stains on the surface;
S2: Preparing the braided tube 22: three-dimensionally braiding a plurality of fiber filaments outside the inner layer tube 21 through a three-dimensional braiding machine to form the braided tube 22;
S3: Preparing the outer layer tube 23: welding the outer layer tube 23 outside the inner layer tube 21 braided with the braided tube 22 by hot melting or heat shrinking, and compounding to form the sheath body 1;
S4: Processing tear grooves 6: cutting tear grooves 6 on the surface of the sheath body 1 through high-precision mechanical cutting technology or laser cutting technology;
S5: Sheathing: tightly wrapping the film 5 on the outside of the sheath body 1 formed with tear grooves 6 in S4, and forming the final product.

Tear grooves 6 are opened on the surface of the composite assembled sheath body 1, and then the film 5 is tightly wrapped outside the sheath body 1 with the tear grooves 6 opened. The film 5 is used to radially limit the sheath body 1, so that the tearable sheath can have sufficient bending support in the radial direction. In step S5, the film 5 is fixedly connected to the surface of the sheath body 1 by any one of bonding, dip coating or welding. In this embodiment, the film is hot-melted and wrapped outside the sheath body 1 by welding. And the outer diameter of the locking cap is equal to the maximum outer diameter of the guiding insertion portion 3, and the outer diameter of the tearable sheath wrapped with the film 5 is equal to the outer diameter of the locking cap, so that the tearable sheath maintains a high degree of concentricity in the overall length direction, which is convenient for the smooth insertion of the tearable sheath.

The above are only preferred embodiments of the present invention, so all equivalent changes or modifications made to the structure, features and principles described in the scope of the present invention's patent application shall be included in the scope of the present invention's patent application.

1. A braided reinforced tearable sheath structure, comprising a sheath body (1), wherein: the sheath body (1) comprises a sleeve portion (2), a guiding insertion portion (3) is provided at the head end of the sleeve portion (2), and a connector (4) is provided at the tail end of the sleeve portion (2);
wherein the sleeve portion (2) comprises an inner tube (21), a braided tube (22), and an outer tube (23) sequentially arranged from the inside to the outside; wherein the sheath body (1) is provided with a plurality of tear grooves (6) along the length direction; each of the tear grooves (6) extends to both ends of the sheath body (1); and the sheath body (1) is further sealed and wrapped with at least one layer of tearable film (5);
wherein the inner side of the tear groove (6) is in communication with the inner cavity of the sleeve portion (2), and the outer side of the tear groove (6) penetrates to the surface of the sheath body (1); and
wherein the tear groove (6) is spirally arranged around the surface of the sheath body (1); or
the tear groove (6) is linearly arranged on the surface of the sheath body (1).

2. The braided reinforced tearable sheath structure according to claim 1, wherein: the outer diameter of the guiding insertion portion (3) gradually decreases from the end close to the sleeve portion (2) toward the end away from the sleeve portion (2).

3. The braided reinforced tearable sheath structure according to claim 1, wherein: the connector (4) is configured as a locking cap for externally connecting an expander.

4. The braided reinforced tearable sheath structure according to claim 1, wherein: the film (5) comprises a tubular polymer heat-shrinkable film, and the material of the polymer heat-shrinkable film is any one of TPE, PO, PVC, PEBAX, PET or silicone rubber.

5. The braided reinforced tearable sheath structure according to claim 1, wherein: the material of the inner tube (21) comprises a self-lubricating polymer, and the material of the self-lubricating polymer is any one of PTFE, PEEK, PE, POM or PI; wherein the braided tube (22) is three-dimensionally braided by a plurality of fiber filaments outside the inner tube (21), and the material of the fiber filaments is any one of polymer wire or metal wire, and the outer tube (23) is a thermoplastic polymer material.

6. A method for manufacturing the braided reinforced tearable sheath structure according to claim 1, comprising the following steps:
S1: preparing the inner tube (21) by alkali washing the surface of the inner tube (21) to remove oil stains on the surface thereof;
S2: preparing the braided tube (22): three-dimensionally braiding a plurality of fiber filaments outside the inner tube (21) through a three-dimensional braiding machine to form the braided tube (22);
S3: preparing the outer tube (23): welding the outer tube (23) outside the inner tube (21) braided with the braided tube (22) by means of hot melting or heat shrinking, and compounding to form the sheath body (1);
S4: processing the tear groove (6): cutting the tear groove (6) on the surface of the sheath body (1) through high-precision mechanical cutting technology;
S5: film coating: tightly wrapping the film (5) on the outside of the sheath body (1) having the tear groove (6) formed in S4, and forming a final product.

7. The method for manufacturing the braided reinforced tearable sheath structure according to claim 6, wherein: in step S5, the film (5) is fixedly connected to the surface of the sheath body (1) by any one of bonding, dip coating or fusion welding.
